(19) United States
(12) Patent Application Publication
(10) Pub. No.: US 2023/0380746 A1
(43) Pub. Date: Nov. 30, 2023

(54) CATHETER END EFFECTOR PROVIDING MAPPING GRID WITH HIGH DENSITY ELECTRODE ARRAY AND STRAIN REDUCTION

(71) Applicant: Biosense Webster (Israel) Ltd., Yokneam (IL)

(72) Inventors: Pieter Emmelius VAN NIEKERK, Rancho Santa Margarita, CA (US); Dustin R. TOBEY, San Dimas, CA (US); Amar PATEL, Irvine, CA (US); Babak EBRAHIMI, Lake Forest, CA (US); John Christian KNUDSON, Anaheim, CA (US)

(21) Appl. No.: 18/303,404

(22) Filed: Apr. 19, 2023

Related U.S. Application Data
(60) Provisional application No. 63/434,273, filed on Dec. 21, 2022.
(60) Provisional application No. 63/347,381, filed on May 31, 2022.

(51) Int. Cl. A61B 5/287; A61B 5/367; A61B 5/00

(52) U.S. Cl. CPC A61B 5/287; A61B 5/367; A61B 18/1492; A61B 5/6858; A61B 5/6856

(57) ABSTRACT

The disclosed technology includes a catheter for electrophysiology applications. The catheter can comprise a shaft extending along a longitudinal axis to a distal end and an end effector coupled to the distal end of the shaft. The end effector can include a plurality of loop members. Each loop member of the plurality of loop members can include a corresponding stress distribution node positioned at a distal portion of the respective loop member and a plurality of electrodes affixed to the plurality of loop members.

### CLAIM OF PRIORITY

This application claims the benefit of priority under 35 U.S.C. § 119 to U.S. Provisional Patent Application No. 63/347,381 filed on May 31, 2022 (Attorney Docket No. BIO6709USPSP1), and U.S. Provisional Patent Application No. 63/434,273 filed on Dec. 21, 2022 (Attorney Docket No. BIO6709USPSP2), each of which is hereby incorporated by reference as if set forth in full herein.

### BACKGROUND

Cardiac arrhythmia, such as atrial fibrillation, occurs when regions of cardiac tissue abnormally conduct electric signals to adjacent tissue, thereby disrupting the normal cardiac cycle and causing asynchronous rhythm. Important sources of undesired signals are located in the tissue region, for example, one of the atria or one of the ventricles. Regardless of the sources, unwanted signals are conducted elsewhere through heart tissue where they can initiate or continue arrhythmia.

Procedures for treating arrhythmia include surgically disrupting the origin of the signals causing the arrhythmia, as well as disrupting the conducting pathway for such signals. More recently, it has been found that by mapping the electrical properties of the endocardium and the heart volume, and selectively ablating cardiac tissue by application of energy, it is possible to cease or modify the propagation of unwanted electrical signals from one portion of the heart to another. The ablation process destroys the unwanted electrical pathways by formation of non-conducting lesions.

In this two-step procedure—mapping followed by ablation—electrical activity at points in the heart is typically sensed and measured by advancing a catheter containing one or more electrical sensors into the heart and acquiring data at a multiplicity of points. These data are then utilized to select the target areas at which ablation is to be performed.

For greater mapping resolution, it is desirable for a mapping catheter to provide very high-density signal maps through the use of a multitude of electrodes sensing electrical activity within a small area, for example, a square centimeter. For mapping within an atria or a ventricle (for example, an apex of a ventricle), it is desirable for a catheter to collect larger amounts of data signals within shorter time spans. It is also desirable for such a catheter to be adaptable to different tissue surfaces, for example, flat, curved, irregular or nonplanar surface tissue and be collapsible for atraumatic advancement and withdrawal through a patient's vasculature.

### SUMMARY

Various embodiments described herein allow high density mapping and/or ablation of tissue surface in the heart, including an atrium or a ventricle, by virtue of a catheter for electrophysiology applications. The catheter includes a tubular member and an end effector. The tubular member extends along a longitudinal axis from a proximal portion to a distal portion. The end effector is coupled to the distal portion. The end effector includes first, second and third loop members, each loop member includes two spines and a connector that connects the two spines and the first, second and third loop members are configured so that each connector of each of the first, second and third loop members is in contact with only one connector of the adjacent loop member.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1A illustrates a catheter from an effector at a distal portion of the catheter to the proximal handle;

FIG. 1B is a close up of the distal portion of the end effector;

FIG. 1C is a perspective cut away view of the proximal portion of the end effector;

FIG. 1D is yet another closer view of FIG. 1C with certain components hidden;

FIG. 1E is a cross-sectional view of the distal end of FIG. 1A;

FIG. 2 illustrates an exploded plan view of the underlying frameworks for the end effector of FIG. 1A;

FIG. 3 illustrates a front elevation view of another example of an end effector for use with the catheter of FIG. 1A;

FIG. 3A is a close up of the distal portion of the end effector of FIG. 3, showing stress distribution nodes of the end effector; and

FIG. 3B is a close up of the distal portion of the end effector of FIG. 3, showing a cover positioned over the spines of the end effector.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment. The term “proximal” and “distal” are used to reference location of various components with respect to the handle which is designated as the most proximal to a user operating the handle.

I. Example of a Catheter End Effector

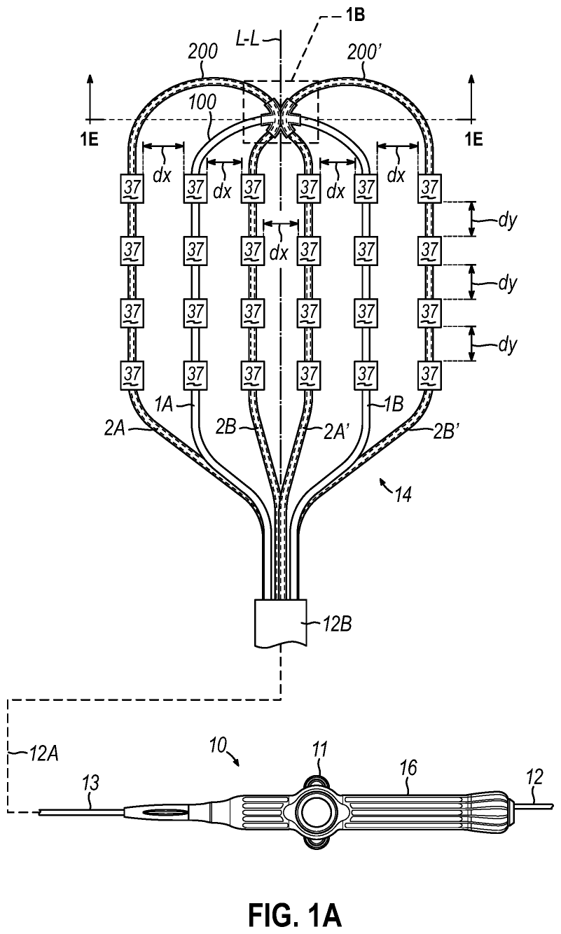

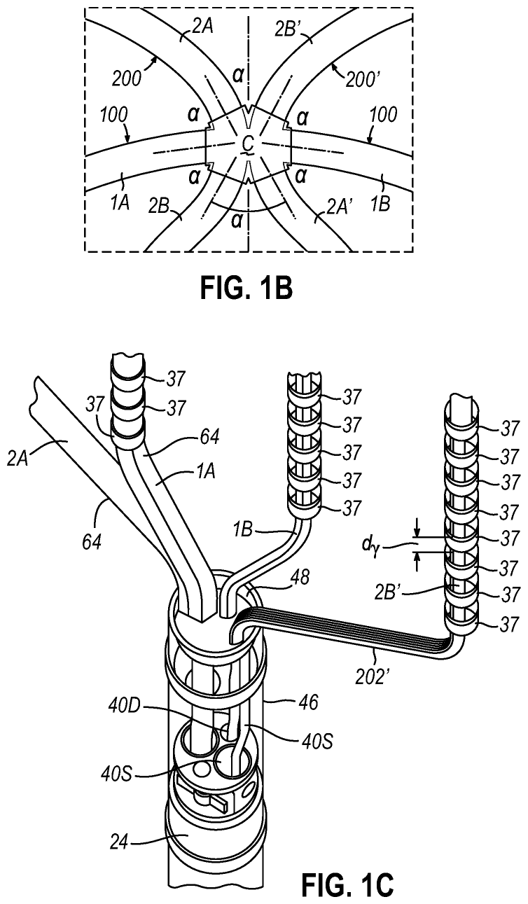

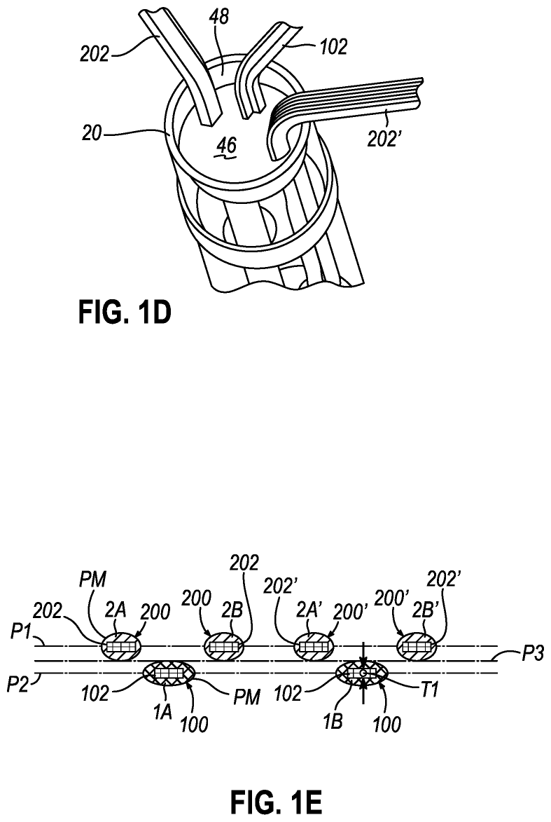

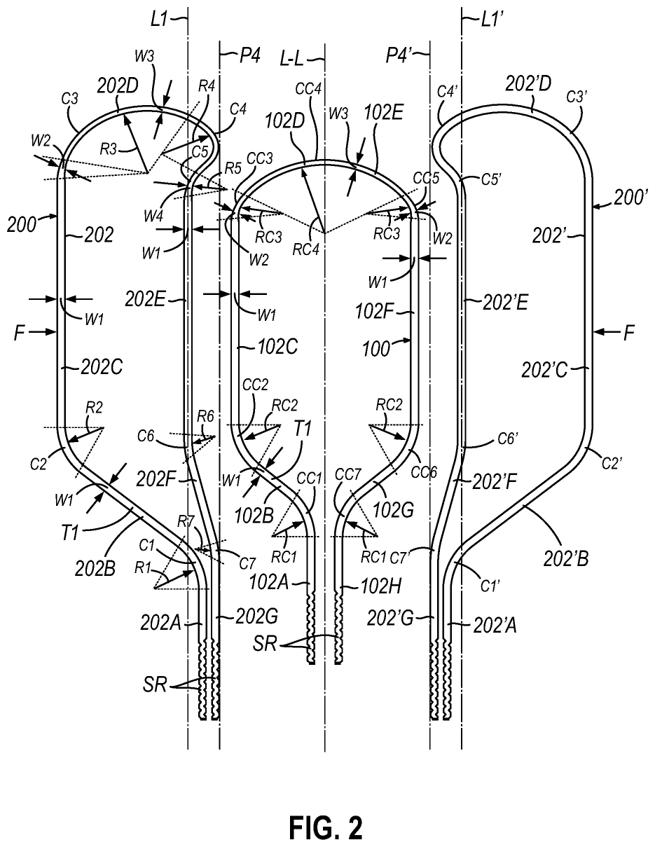

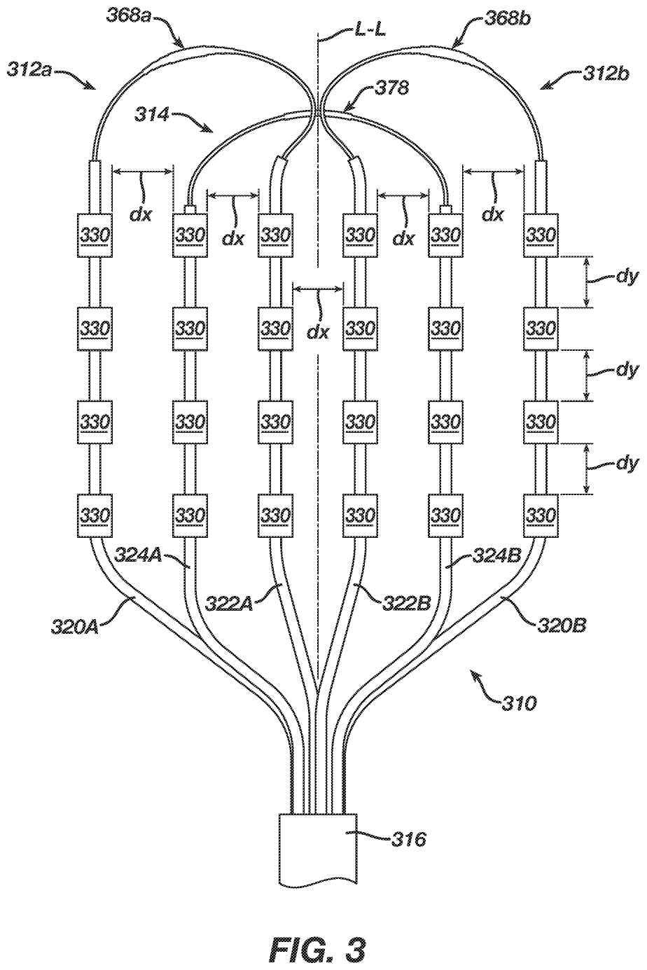

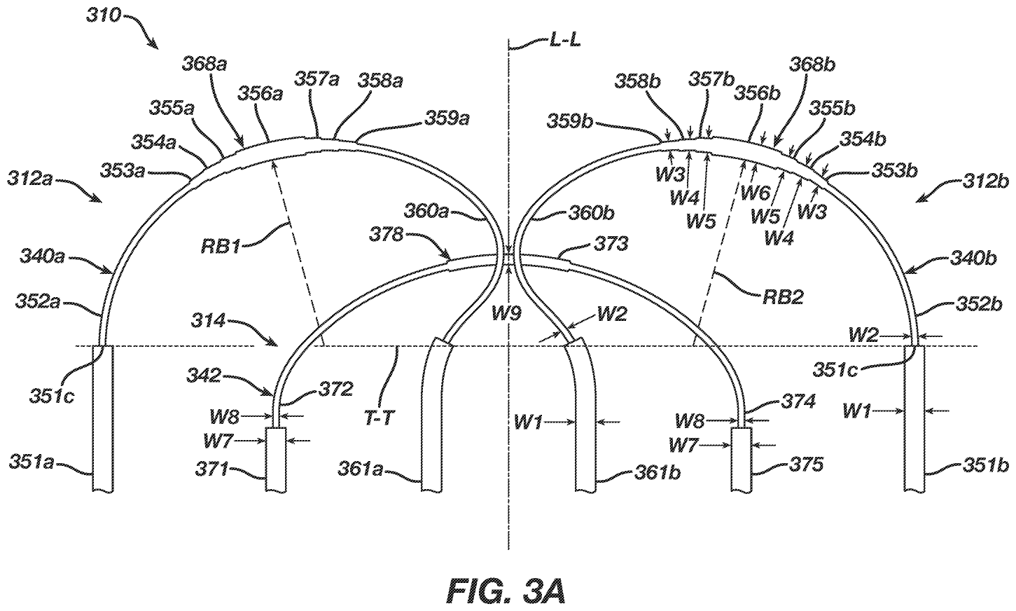

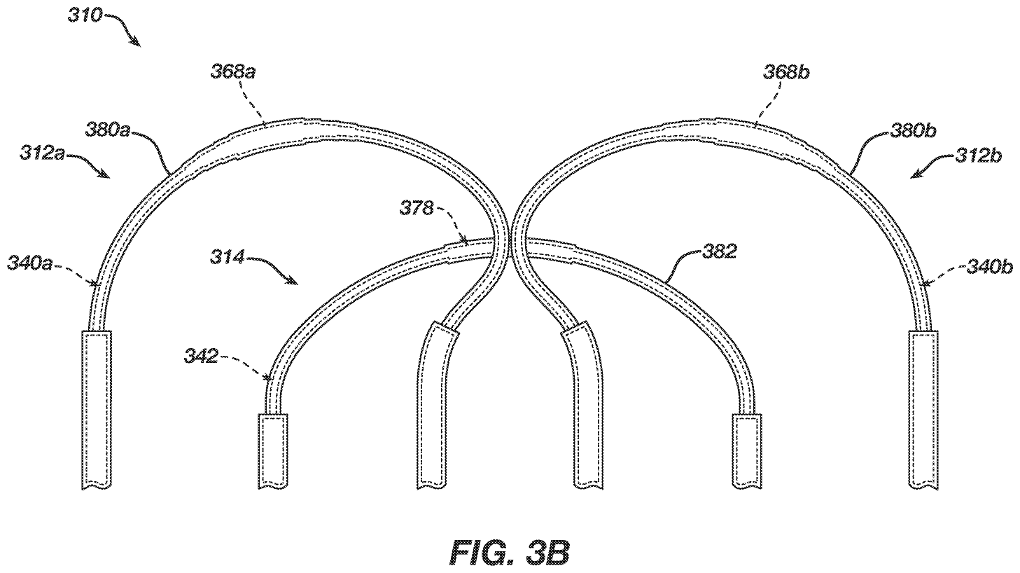

As shown in FIG. 1, the catheter 10 comprises an elongated catheter body 12 disposed inside a separate intermediate tubular section 13, a distal electrode assembly or end effector 14, and a deflection control handle 16 attached to the proximal end of the catheter body 12. In accordance with a feature of the present invention, the effector 14 has a plurality of spines 1A, 1B, 2A, 2B, 3A, 3B that lies on different planes. The intermediate section 13 can extend along a longitudinal axis L-L along a proximal portion 12A to a distal portion 12B before deployment of the end effector 14. One or more impedance sensing electrode can be provided on distal portion 12B (not shown) to allow for location sensing via impedance location sensing technique, as described in U.S. Pat. No. 5,944,022 entitled “Catheter Positioning System,” issued Aug. 31, 1999; U.S. Pat. No. 5,983,126 entitled “Catheter Location System and Method,” issued Nov. 9, 1999; and U.S. Pat. No. 6,445,864, entitled “Dispersion Compensating Optical Fiber,” issued Sep. 3, 2002. The disclosures of each of these references is incorporated herein by reference, in its entirety. Details of the catheter body 16 can be understood from a review of U.S. Pub. No. 2020/0345262, entitled “Mapping Grid with High Density Electrode Array,” published Nov. 5, 2020, including the description to the handle and particularly FIGS. 2D, 2E and 3 in this prior application, the disclosure of which is incorporated by reference herein, in its entirety.

Distal portion (12B) is coupled to an end effector (14) in FIG. 1A. The end effector (14) has three loop members (100), (200) and (200′) with two loop members (200) and (200′) is identical and disposed on either side of central loop member (100). In FIG. 1A, it can be seen that loop (200) is identical to loop (200′) except that loop (200′) is oriented 180 degrees compared to loop (200) with respect to axis (L-L). That is, loop (200) and loop (200′) can be considered to be mirror images of each other. Loop (100) on the other hand is distinct from loops (200) and (200′). The three loops (100, 200 200′) are tied together at the distal end shown in FIG. 1B. In FIG. 1B, spine member (1A) is integral with spine member (1B) as part of loop (100) and spine members (2A, 2B) are integral to loop (200). Similarly, spine members (2A′, 2B′) are integral to loop (200′). These spine members (1A, 1B, 2A, 2B, 2A′, 2B′) cross at a common center (C) such that each spine member (1A, 1B, 2A, 2B, 2A′, 2B′) is angled relative to each of its adjacent neighboring spines (1A, 1B, 2A, 2B, 2A′, 2B′) at a separation angle (α) of approximately 30 degrees. For example, spine (1A) is separated from each of its neighboring spines (2A, 2B) by a respective angle (α).

As shown in FIGS. 1C and 1D, four spines including spines (1A, 1B) of loop (100) and spines (2A, 2B) of loop (200) are shown with the cover (typically polymer such as polyurethane) on spines (1B, 2A, 2B) removed so that structural member (102, 202, 202′) for central loop 100 and outer loops (200, 200′) can be seen. That is, each outer loop (200, 200′) with spine frame (202, 202′) and central loop (100) with frame (102) has a respective elongated structural member (102, 202, 202′) extending through the length of the loop (100, 200, 200′), to and from insert member (20) which is part of tubular portion (46). While the preferred embodiment of the loops is formed from a single unitary material, it is within the scope of this invention that each segment of each loop (as described in FIG. 2) can be discrete components affixed to each other. A proximal portion of each loop structural member (102, 202, 202′) extends into a distal end portion of the connector tubing (46) (which is part of tubular member (12)) and is anchored in the lumen (48) into insert (20). To ensure that the loops (100, 200, 200′) can be compressed into a very small shape for delivery into a vessel, we have devised the outer loops (200, 200′) to have a structural member (202, 202′) in the asymmetric configuration shown in FIG. 2 while structural member (102) for the central loop (100) has the symmetric configuration shown in FIG. 2.

Referring to FIG. 2, structural member (102) for central loop (100) is a unitary structural framework that has been physically manipulated to define different segments. In FIG. 2, it is noted that the central loop (100) includes a continuous central frame (102), in which the continuous central frame (102) may include the first to eighth linear segments (102A, 102B, 102C, 102D, 102E, 102F, 102G, 102H), respectively, with first to seventh curved segments (CC1, CC2, CC3, CC4, CCS, CC6, CC7) disposed between the respective first to eighth linear segments (102A, 102B, 102C, 102D, 102E, 102F, 102G, 102H).

Each of the outer loop members (200, 200′) includes a respective first linear segment (202A, 202′A) connected to a respective second linear segment (202B, 202′B) with a respective first curved segment (C1, C1′), the second linear segment (202B, 202′B) is connected to a respective third linear segment (202C, 202′C) via a respective second curved segment (C2, C2′). The third linear segment (202C, 202′C) is connected to a respective distal curved segment (202D, 202′D) which is connected to a respective fourth linear segment (202E, 202′E) which is connected to a respective fifth linear segment (202F, 202′F) via a respective sixth curved segment (C6, C6′), the fifth linear segment (202F, 202′F) is connected to a respective sixth linear segment (202G, 202′G) via a respective seventh curved segment (C7, C7′). The seventh curved segment (C7, C7′) is becomes the respective final sixth linear segment (202G, 202′G).

In FIG. 2, it can be seen that distal curved segment (202D, 202′D) of each outer loop (200, 200′) may include a respective third curved segment (C3, C3′) extending to a respective fourth curved segment (C4, C4′) which becomes a respective fifth curved segment (C5, C5′) which continues on as the respective fourth linear segment (202E, 202′E). As noted, each of the outer loops (200, 200′) can be from a respective continuous outer frame (202, 202′) that can be formed from multiple pieces but are preferably unitary.

Of note here is that fourth curved segments (C4, C4′) for outer frames (202, 202′) are each configured to protrude towards the longitudinal axis L-L in which the respective fourth curved segment (C4, C4′) extends beyond a respective virtual center line (L1, L1′) of the corresponding fourth linear segment (202E, 202′E) and contiguous (or tangential) to a respective plane (P4, P4′) which is parallel to the corresponding sixth linear segment (202G, 202′G) and the longitudinal axis (L-L). This configuration of the outer loops (200, 200′) is believed to ensure sufficient structural robustness to allow each of the outer loops (202, 202′) to maintain its intended shape when deployed into a much larger planar configuration despite is compressed into a tubular sheath (13) during storage prior to is used in an electrophysiology procedure.

By virtue of this design, the outer frames (202, 202′) are disposed on each side of the central loop frame (102) so that one outer loop frame (202) can be seen as a mirror image of the other outer loop frame (202′). As shown in FIG. 1E, a first virtual plane (P1) extends through centers of the outer loops (200, 200′) and a second plane (P2) parallel to the first plane (P1) extends through the centers of the central loop (100). Note that the outer loops (200, 200′) and the central loop (100) are contiguous to a third plane (P3) disposed between first and second planes (P1, P2).

The central and outer frames (102, 202, 202′) each have a generally rectangular cross section of varying dimensions with a maximum width of about 0.25 mm and a minimum width of about 0.1 mm with a thickness of about 0.1 mm. In particular, the width (W1) of the central frame (102) and outer frames (202, 202′) is approximately 0.2 mm to 0.3 mm for a cross-sectional area of approximately 0.02 mm-squared to 0.03 mm-squared. Width (W2) of each frame (102, 202, 202′) is a tapering width (W2) that tapers continuously from the width (W1) to about 0.1 mm for width (W3) for a tapering cross-sectional area starting from 0.03 mm-squared to 0.01 mm-squared. Width (W3) is approximately 0.1 mm for a cross-sectional area of about 0.01 mm-squared. Width (W4) for the outer frames (202, 202′) is an increasing width that increases from width (W3) (about 0.1 mm) to a width of any value in the range of approximately 0.2 mm to approximately 0.3 mm for increasing cross-sectional areas from 0.01 mm-squared to 0.03 mm-squared. Viewing the width of the distal end curves (202D, 202′D) another way, it can be seen that the width (W3) is approximately half of the width (W1) for the main portions (202C, 202E, 202′C, 202′E) of the respective loop frame (202, 202′). At its maximum, width (W4) is now the same as width (W1) for the remainder of the segments all the way to the respective end segment (202G, 202′G). End segments (202G, 202′G) can each be provided with serrations (SR) to allow the end segments (202G, 202′G) to be locked to each other or overmolded into tubular portion (46). The frames (102, 202 and 202′) each have the same thickness (T1) (FIG. 1E) of approximately 0.1 mm. The frames (102, 202 and 202′) can each include a biocompatible metal such as stainless steel, cobalt chromium or nitinol.

In FIG. 2, radius of curvature (R1) and/or radius of curvature (R2) may be about 2 mm; radius of curvature (R3) may be about 3 mm; radius of curvature (R4) and/or radius of curvature (R5) may be about 1.5 mm; radius of curvature (R6) and/or radius of curvature (R7) maybe about 3 mm; radius of curvature (RC1) and/or radius of curvature (RC2) may be about 3 mm; radius of curvature (RC3) may be about 1.5 mm; and/or radius of curvature (RC4) may be about 4 mm.

While the frames (102, 202, 202′) are described with rectangular cross-sections, it should be noted that various non-rectangular (i.e., circular, ovoid, or semi-circular cross-sections) can be used as long as the cross-sections have the same cross-sectional areas as noted above and the described cross-sectional areas are within the scope of this invention.

As shown in FIG. 1A, the completed end effector (14) has a cover in the form of a polymer material (PM) disposed over each of the continuous central and outer frames (102, 202, 202′) with a plurality of electrodes (37) disposed on an outer surface of the polymer material (PM). In FIG. 1B, it can be seen that the central loop (100) and the outer loops (200, 200′) are physically connected at a distal location (C) of the central frame (102) proximate the longitudinal axis (L-L) such that each of the distal portions (1A, 1B, 2A, 2B, 2A′, 2B′) are subtended to its neighboring loops by separation angle (α) of approximately 30 degrees. In particular, distal portion (1A) of central loop (100) is separated by its neighboring distal portion (2A) of outer loop (200) and distal portion (2B) of outer loop (200) with approximately the same angle (α). Similarly, distal portion (1B) of central loop (100) is separated by angle (α) from its neighboring distal portion (2A′) of outer loop (200′) and distal portion (2B′) of outer loop (200′) by approximately the same angle (α). The physical connection (C) can be in the form of a suture or reflowing of the polymer material PM so that all the members are physically joined together due to the heating and reflowing of the polymer.

Referring back to FIG. 1A, it can be seen that the plurality of electrodes (37) are disposed over the outer loops (200, 200′) and the central loop (100) so that the electrodes (37) are spaced apart over a distance from its adjacent neighboring electrodes (37) (as measured from center of one electrode (37) to center of another electrode (37)) by a first distance (dy) along the longitudinal axis (L-L) and a second distance (dx) along an axis transverse to the longitudinal axis (L-L). While the distances (dx, dy) can be different or unequal from each other, the preference is to configure the spacings (dx, dy) so that the first and second distances (dy, dx) are approximately equal to each other.

II. Example of Alternative Loop Configuration for a Catheter End Effector

In some procedures, it may be desirable to selectively house end effector (14) within tubular sheath (13) and selectively deploy end effector (14) from tubular sheath (13), such that loops (100, 200, 200′) are elastically compressed to a collapsed state (not shown) when end effector (14) is housed within tubular sheath (13), and such that loops (100, 200, 200′) resiliently expand from the collapsed state to an expanded state in which loops (100, 200, 200′) assume the configurations shown in FIG. 1A when end effector (14) is deployed from tubular sheath (13). In addition, or alternatively, it may be desirable to direct end effector (14) into tubular sheath (13) using an insertion tool (not shown). Therefore, it may be desirable to configure loops (100, 200, 200′) to distribute stress across loops (100, 200, 200′) in a predetermined manner that allows loops (100, 200, 200′) to maintain their elasticity and avoid yielding to plastic deformation, which may otherwise occur when the outermost spines (2A, 2B′) and/or distal segments (102D, 102E, 202D, 202′D) of loops (100, 200, 200′) are subjected to various stress concentrations during repeated cycles of deployment and housing of end effector (14) relative to tubular sheath (13) and/or repeated cycles of being directed into tubular sheath (13) via the insertion tool.

In this regard, loops (100, 200, 200′) may each experience two deformation modes upon repeated cycles of deployment and housing: in-plane deformation (also referred to as planar deformation) and out-of-plane deformation. Some cases of planar deformation may include inward bowing of the two outermost spines (2A, 2B′) toward each other such that the two outermost spines (2A, 2B′) collectively define an hourglass shape, which may occur when the two outermost spines (2A, 2B′) experience substantially high stress concentrations at or near their respective middle portions upon entry into tubular sheath (13). Such deformation may be referred to as “hourglass deformation.” In addition, or alternatively, some cases of planar deformation may include creasing of any one or more of the distal segments (102D, 102E, 202D, 202′D). Such deformation may be referred to as “distal curve creasing.” Some cases of out-of-plane deformation may include inward folding of the two outer loops (200, 200′) toward each other around the central loop (100), such as due to the two outermost spines (2A, 2B′) is parts of two separate loops (200, 200′) which can move independently of each other, deform, and rotate to curl in the direction of collapse. Such deformation may be referred to as “paddle folding.”

Distributing stress across loops (100, 200, 200′) in a predetermined manner that minimizes or eliminates undesirable stress concentrations may inhibit the aforementioned modes of deformation from occurring, such that loops (100, 200, 200′) may consistently and reliably expand from the collapsed state to the desired expanded state in which electrodes (37) may be spaced apart from each other by predefined distances, such as distances (dx, dy) described above. It will be appreciated that avoiding inadvertent deviations from such predefined distances (dx, dy) when in the expanded state may facilitate consistent and reliable functionality of electrodes (37).

In some procedures, it may be desirable to increase the area over which stresses are distributed across various portions of loops (100, 200, 200′) to thereby reduce or eliminate the amounts of strain experienced by each loop (100, 200, 200′). For example, it may be desirable to increase the area over which stresses are distributed across the portions of loops (100, 200, 200′) that are subjected to the highest amounts of stresses during normal use. In this regard, it will be appreciated that the retraction of end effector (14) relative to tubular sheath (13) and/or the direction of end effector (14) into tubular sheath (13) via the insertion tool may cause the highest amounts of stresses experienced by loops (100, 200, 200′) at or near the respective distal segments (102D, 102E, 202D, 202′D), thereby resulting in relatively high strain points at or near the respective distal segments (102D, 102E, 202D, 202′D). Such high strain points may ultimately result in permanent deformation of the underlying frames (102, 202 and 202′). Increasing the area over which stresses are distributed across distal segments (102D, 102E, 202D, 202′D) of loops (100, 200, 200′) may inhibit such permanent deformation from occurring, such that loops (100, 200, 200′) may consistently and reliably expand from the collapsed state to the desired expanded state in which electrodes (37) may be spaced apart from each other by predefined distances, such as distances (dx, dy) described above. As mentioned above, avoiding inadvertent deviations from such predefined distances (dx, dy) when in the expanded state may facilitate consistent and reliable functionality of electrodes (37).

FIGS. 3-3B depict an example of an end effector (310) having such a configuration, and which may be incorporated into catheter (10) in place of end effector (14). End effector (310) may be similar to end effector (14) described above except as otherwise described below. In this regard, end effector (310) of this example includes first and second outer loop members (312a, 312b) and an inner loop member (314), each extending distally from a base member or shaft (316), which itself extends along longitudinal axis (L-L). Loop members (312a, 312b, 314) may also be referred to as paddles, loops, and/or electrode loop assemblies.

In the example shown, first and second outer loop members (312a, 312b) are substantially identical to each other and are disposed on either side of longitudinal axis (L-L), at least partially radially outwardly of third or central loop member (314). More particularly, outer loop members (312a, 312b) each include a corresponding outermost spine member (320a, 320b) and a corresponding innermost spine member (322a, 322b), while inner loop member (314) includes a pair of intermediate spine members (324a, 324b). Spine members (320a, 320b, 322a, 322b, 324a, 324b) may also be referred to as spines. In this regard, outermost spines (320a, 320b) are positioned radially outwardly of intermediate spines (324a, 324b) with respect to longitudinal axis (L-L), and innermost spines (322a, 322b) are positioned radially inwardly of intermediate spines (324a, 324b) with respect to longitudinal axis (L-L), at least within the frame of reference of FIG. 3. It will be appreciated that first outermost spine member (320a) is integral with first innermost spine member (322a) as part of first outer loop (312a) and that second outermost spine member (320b) is integral with second innermost spine member (322b) as part of second outer loop (312b). Similarly, first intermediate spine member (324a) is integral with second intermediate spine member (324b) as part of inner loop (314).

Loop members (312a, 312b, 314) also each include a corresponding plurality of sensing electrodes (330) secured to the respective spine members (320a, 320b, 322a, 322b, 324a, 324b). Electrodes (330) may be configured to provide electrophysiology (EP) mapping, such as to identify tissue regions that should be targeted for ablation. For example, electrodes (330) may monitor electrical signals emanating from conductive endocardial tissues to pinpoint the location of aberrant conductive tissue sites that are responsible for an arrhythmia. By way of example only, electrodes (330) may be configured and operable in accordance with at least some of the teachings of U.S. Pub. No. 2020/0345262, entitled “Mapping Grid with High Density Electrode Array,” published Nov. 5, 2020, the disclosure of which is incorporated by reference herein, in its entirety.

In the example shown, each loop member (312a, 312b, 314) includes a corresponding structural member (340a, 340b, 342). In this regard, each outer loop member (312a, 312b) has a corresponding elongated structural member (340a, 340b) extending continuously through the length of the respective outer loop member (312a, 312b) to and from base member (316), while inner loop member (314) has an elongated structural member (342) extending continuously through the length of inner loop member (314) to and from base member (316). Structural members (340a, 340b, 342) may also be referred to as loop structural members, spine frames, or frames. Structural members (340a, 340b, 342) can each include a biocompatible metal such as stainless steel, cobalt chromium, or nitinol.

As best shown in FIG. 3A, structural members (340a, 340b) of outer loops (312a, 312b) are each formed as a unitary structural framework defining respective pluralities of segments (351a, 351b, 352a, 352b, 353a, 353b, 354a, 354b, 355a, 355b, 356a, 356b, 357a, 357b, 358a, 358b, 359a, 359b, 360a, 360b, 361a, 361b) having varying widths (W1, W2, W3, W4, W5, W6); and structural member (342) of inner loop (314) is formed as a unitary structural framework defining a plurality of segments (371, 372, 373, 374, 375) having varying widths (W7, W8, W9).

More particularly, structural members (340a, 340b) of outer loops (312a, 312b) each include a respective first segment (351a, 351b) extending distally from base member (316), and having a first width (W1); a respective second segment (352a, 352b) extending distally and/or laterally inwardly from the respective first segment (351a, 351b), and having a second width (W2) less than the first width (W1) of first segments (351a, 351b); a respective third segment (353a, 353b) extending distally and/or laterally inwardly from the respective second segment (352a, 352b), and having a third width (W3) greater than the second width (W2) of second segments (352a, 352b); a respective fourth segment (354a, 354b) extending distally and/or laterally inwardly from the respective third segment (353a, 353b), and having a fourth width (W4) greater than the third width (W3) of third segments (353a, 353b); a respective fifth segment (355a, 355b) extending distally and/or laterally inwardly from the respective fourth segment (354a, 354b), and having a fifth width (W5) greater than the fourth width (W4) of fourth segments (354a, 354b); a respective sixth segment (356a, 356b) extending distally and/or laterally inwardly from the respective fifth segment (355a, 355b), and having a sixth width (W6) greater than the fifth width (W5) of fifth segments (355a, 355b); a respective seventh segment (357a, 357b) extending proximally and/or laterally inwardly from the respective sixth segment (356a, 356b), and having the same fifth width (W5) as fifth segments (355a, 355b); a respective eighth segment (358a, 358b) extending proximally and/or laterally inwardly from the respective seventh segment (357a, 357b), and having the same fourth width (W4) as fourth segments (354a, 354b); a respective ninth segment (359a, 359b) extending proximally and/or laterally inwardly from the respective eighth segment (358a, 358b), and having the same third width (W3) as third segments (353a, 353b); a respective tenth segment (360a, 360b) extending proximally and/or laterally inwardly from the respective ninth segment (359a, 359b) (and curving laterally outwardly in the example shown), and having the same second width (W2) as second segments (352a, 352b); and a respective eleventh segment (361a, 361b) extending proximally from the respective tenth segment (360a, 360b) to base member (316), and having the same first width (W1) as first segments (351a, 351b). As discussed in greater detail below, the transitions between adjacent widths, such as the transition from the first width (W1) to the second width (W2), may be gradual (e.g., tapered).

Structural member (342) of inner loop (314) includes a first segment (371) extending distally from base member (316), and having a seventh width (W7); a second segment (372) extending distally and/or laterally inwardly from first segment (371), and having an eighth width (W8) less than the seventh width (W7) of first segment (371); a third segment (373) extending laterally across the longitudinal axis (L-L) from second segment (372), and having a ninth width (W9) greater than the eighth width (W8) of second segment (372); a fourth segment (374) extending proximally and/or laterally outwardly from third segment (373), and having the same eighth width (W8) as second segment (372); and a fifth segment (375) extending proximally from fourth segment (374) to base member (316), and having the same seventh width (W7) as first segment (371). In some versions, the seventh width (W7) may be the same as the first width (W1); the eighth width (W8) may be the same as the second width (W2); and/or the ninth width (W9) may be the same as the sixth width (W6).

The first width (W1) may be about 0.01 inch, for example; the second width (W2) may be about 0.005 inch, for example; the third width (W3) may be about 0.00525 inch, for example; the fourth width (W4) may be about 0.00550 inch, for example; the fifth width (W5) may be about 0.00575 inch, for example; and/or the sixth width (W6) may be about 0.006 inch, for example. Thus, the sixth width (W6) may be about 20% greater than the second width (W2) in some versions.

As shown, third, fourth, fifth, sixth, seventh, eighth, and ninth segments (353a, 353b, 354a, 354b, 355a, 355b, 356a, 356b, 357a, 357b, 358a, 358b, 359a, 359b) are positioned at distal portions of the respective outer loops (312a, 312b), at which the highest amounts of stress may be experienced by outer loops (312a, 312b) during normal use. In some versions, sixth segments (356a, 356b) may be positioned at or near the distal-most portions of the respective outer loops (312a, 312b). Due to the increased magnitudes of the third, fourth, fifth, and sixth widths (W3, W4, W5, W6) relative to the second width (W2), third, fourth, fifth, sixth, seventh, eighth, and ninth segments (353a, 353b, 354a, 354b, 355a, 355b, 356a, 356b, 357a, 357b, 358a, 358b, 359a, 359b) may provide an increased area over which the stresses are distributed across the distal portion of the respective outer loop (312a, 312b) (e.g., at least by comparison to scenarios in which the third, fourth, fifth, and/or sixth widths (W3, W4, W5, W6) are substantially equal to or less than the second width (W2)). In this manner, third, fourth, fifth, sixth, seventh, eighth, and ninth segments (353a, 353b, 354a, 354b, 355a, 355b, 356a, 356b, 357a, 357b, 358a, 358b, 359a, 359b) of each outer loop (312a, 312b) may collectively define a corresponding stress distribution node (368a, 368b) of the respective outer loop (312a, 312b). In some versions, stress distribution node (368a, 368b) of each outer loop (312a, 312b) may be defined by more or less than third, fourth, fifth, sixth, seventh, eighth, and ninth segments (353a, 353b, 354a, 354b, 355a, 355b, 356a, 356b, 357a, 357b, 358a, 358b, 359a, 359b). For example, stress distribution node (368a, 368b) of each outer loop (312a, 312b) may be defined by only the respective sixth segment (356a, 356b), with third, fourth, fifth, seventh, eighth, and ninth segments (353a, 353b, 354a, 354b, 355a, 355b, 357a, 357b, 358a, 358b, 359a, 359b) being omitted such that sixth segments (356a, 356b) are interposed directly between the respective second and tenth segments (352a, 352b, 360a, 360b).

Likewise, third segment (373) is positioned at a distal portion of inner loop (314), at which the highest amounts of stress may be experienced by inner loop (314) during normal use. In some versions, third segment (373) may be positioned at or near the distal-most portion of inner loop (314). Due to the increased magnitude of the ninth width (W9) relative to the eighth width (W8), third segment (373) may provide an increased area over which the stresses are distributed across the distal portion of inner loop (314) (e.g., at least by comparison to scenarios in which the ninth width (W9) is substantially equal to or less than the eighth width (W8)). In this manner, third segment (373) of inner loop (314) may define a corresponding stress distribution node (378) of inner loop (314).

In some versions, first segments (351a, 351b) of outer loops (312a, 312b) may have a first thickness (not shown), and eleventh segments (361a, 361b) of outer loops (312a, 312b) may have the same first thickness as first segments (351a, 351b). The first thickness may be about 0.01 inch, for example. In addition, or alternatively, second segments (352a, 352b) of outer loops (312a, 312b) may have a second thickness (not shown) less than the first thickness, and any one or more of third segments (353a, 353b), fourth segments (354a, 354b), fifth segments (355a, 355b), sixth segments (356a, 356b), seventh segments (357a, 357b), eighth segments (358a, 358b), ninth segments (359a, 359b), and/or tenth segments (360a, 360b) of outer loops (312a, 312b) may have the same second thickness as second segments (352a, 352b). The second thickness may be about 0.005 inch, for example. First segment (371) and fifth segment (375) of inner loop (314) may have the same first thickness as first segments (351a, 351b) and eleventh segments (361a, 361b) of outer loops (312a, 312b); and second, third, and fourth segments (372, 373, 374) of inner loop (314) may have the same second thickness as second, third, fourth, fifth, sixth, seventh, eight, ninth, and/or tenth segments (352a, 352b, 353a, 353b, 354a, 354b, 355a, 355b, 356a, 356b, 357a, 357b, 358a, 358b, 359a, 359b, 360a, 360b) of outer loops (312a, 312b).

In the example shown, the first width (W1) of first segments (351a, 351b) and eleventh segments (361a, 361b) is substantially equal to the first thickness of first segments (351a, 351b) and eleventh segments (361a, 361b), such that first segments (351a, 351b) and eleventh segments (361a, 361b) each have a generally square cross-sectional shape; the second width (W2) of second segments (352a, 352b) and tenth segments (360a, 360b) is substantially equal to the second thickness of second segments (352a, 352b) and tenth segments (360a, 360b), such that second segments (352a, 352b) and tenth segments (360a, 360b) each have a generally square cross-sectional shape; the third width (W3) of third segments (353a, 353b) and ninth segments (359a, 359b) is substantially greater than the second thickness of third segments (353a, 353b) and ninth segments (359a, 359b), such that third segments (353a, 353b) and ninth segments (359a, 359b) each have a generally rectangular cross-sectional shape; the fourth width (W4) of fourth segments (354a, 354b) and eighth segments (358a, 358b) is substantially greater than the second thickness of fourth segments (354a, 354b) and eighth segments (358a, 358b), such that fourth segments (354a, 354b) and eighth segments (358a, 358b) each have a generally rectangular cross-sectional shape; the fifth width (W5) of fifth segments (355a, 355b) and seventh segments (357a, 357b) is substantially greater than the second thickness of fifth segments (355a, 355b) and seventh segments (357a, 357b), such that fifth segments (355a, 355b) and seventh segments (357a, 357b) each have a generally rectangular cross-sectional shape; and the sixth width (W6) of sixth segments (356a, 356b) is greater than the second thickness of sixth segments (356a, 356b), such that sixth segments (356a, 356b) each have a generally rectangular cross-sectional shape.

As shown, each transition between adjacent pairs of segments (351a, 351b, 352a, 352b, 353a, 353b, 354a, 354b, 355a, 355b, 356a, 356b, 357a, 357b, 358a, 358b, 359a, 359b, 360a, 360b, 361a, 361b, 371, 372, 373, 374, 375) may have a stepped configuration. In some other versions, the transitions between any one or more adjacent pairs of segments (351a, 351b, 352a, 352b, 353a, 353b, 354a, 354b, 355a, 355b, 356a, 356b, 357a, 357b, 358a, 358b, 359a, 359b, 360a, 360b, 361a, 361b, 371, 372, 373, 374, 375) may have continuously tapered configurations. For example, structural members (340a, 340b) may each taper outwardly (relative to a central axis of the respective structural member (340a, 340b)) from the respective second segment (352a, 352b) to the respective third segment (353a, 353b); from the respective third segment (353a, 353b) to the respective fourth segment (354a, 354b); from the respective fourth segment (354a, 354b) to the respective fifth segment (355a, 355b); and/or from the respective fifth segment (355a, 355b) to the respective sixth segment (356a, 356b). In addition, or alternatively, structural members (340a, 340b) may each taper inwardly (relative to a central axis of the respective structural member (340a, 340b)) from the respective sixth segment (356a, 356b) to the respective seventh segment (357a, 357b); from the respective seventh segment (357a, 357b) to the respective eighth segment (358a, 358b); from the respective eighth segment (358a, 358b) to the respective ninth segment (359a, 359b); and/or from the respective ninth segment (359a, 359b) to the respective tenth segment (360a, 360b). In some versions, structural members (340a, 340b) may each taper outwardly (relative to a central axis of the respective structural member (340a, 340b)) continuously from the respective second segment (352a, 352b) to the respective sixth segment (356a, 356b), and/or may each taper inwardly (relative to a central axis of the respective structural member (340a, 340b)) continuously from the respective sixth segment (356a, 356b) to the respective tenth segment (360a, 360b).

In the example shown, the third, fourth, fifth, sixth, and ninth widths (W3, W4, W5, W6, W9) are each defined by the respective structural member (340a, 340b, 342) itself. In some other versions, one or more sleeves (not shown) may be applied over structural members (340a, 340b, 342) to define any one or more of the third, fourth, fifth, sixth, and/or ninth widths (W3, W4, W5, W6, W9). In this manner, such sleeves may contribute to defining the respective stress distribution nodes (368a, 368b, 378). It will be appreciated that such sleeves may comprise the same material as that of the respective structural member (340a, 340b, 342) and/or a different material from that of the respective structural member (340a, 340b, 342).

While stress distribution nodes (368a, 368b) of outer loops (312a, 312b) in the present example are defined by third, fourth, fifth, sixth, seventh, eighth, and ninth segments (353a, 353b, 354a, 354b, 355a, 355b, 356a, 356b, 357a, 357b, 358a, 358b, 359a, 359b) via the increased magnitudes of the third, fourth, fifth, and sixth widths (W3, W4, W5, W6) relative to the second width (W2), it will be appreciated that stress distribution nodes (368a, 368b) of outer loops (312a, 312b) may additionally or alternatively be defined by increased magnitudes of the thickness of third, fourth, fifth, sixth, seventh, eighth, and ninth segments (353a, 353b, 354a, 354b, 355a, 355b, 356a, 356b, 357a, 357b, 358a, 358b, 359a, 359b) relative to that of second and tenth segments (352a, 352b, 360a, 360b). For example, rather than each having the same second thickness as second and tenth segments (352a, 352b, 360a, 360b), third, fourth, fifth, sixth, seventh, eighth, and ninth segments (353a, 353b, 354a, 354b, 355a, 355b, 356a, 356b, 357a, 357b, 358a, 358b, 359a, 359b) may have respective thicknesses that are substantially greater than the second thickness, such as third, fourth, fifth, and sixth thicknesses with magnitudes similar to those described above for the third, fourth, fifth, and sixth widths (W3, W4, W5, W6), respectively.

Each of the stress distribution nodes (368a, 368b) may have its thickest portion (i.e., largest cross-sectional area) located at a respective distance (RB1, RB2) from a transverse axis (T-T) (which is perpendicular to the longitudinal axis (L-L)). The transverse axis (T-T) intersects structural member (340a) at a location where curved segment (352a) and linear segment (351a) comes together at interface (351c). The distance (RB1) may be from about 2 mm to about 4 mm, such as about 3 mm. The distance (RB2) may have the same range as distance (RB1).

Similarly, while stress distribution node (378) of inner loop (314) in the present example is defined by third segment (373) via the increased magnitude of the ninth width (W9) relative to the eighth width (W8), it will be appreciated that stress distribution node (378) of inner loop (314) may additionally or alternatively be defined by an increased magnitude of the thickness of third segment (373) relative to that of second and fourth segments (372, 374). For example, rather than each having the same second thickness as second and fourth segments (372, 374), third segment (373) may have a thickness that is substantially greater than the second thickness, such as a thickness with a magnitude similar to that described above for the ninth width (W9).

As shown in FIG. 3B, each loop member (312a, 312b, 314) may further include a corresponding cover (380a, 380b, 382), with the corresponding structural member (340a, 340b, 342) (including the corresponding stress distribution node (368a, 368b, 370) underlying the corresponding cover (380a, 380b, 382) and with the corresponding plurality of electrodes (330) disposed on an outer surface of the corresponding cover (380a, 380b, 382). Covers (380a, 380b, 382) may each be electrically insulative, and/or may each include a polymeric material such as polyurethane, for example. By way of example only, each cover (380a, 380b, 382) may be provided as a coating, as an overmold, or in some other suitable fashion.

III. Examples of Combinations That Are Within the Scope of the Inventions

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

EXAMPLE 1 can be a catheter (14 in FIG. 1) intended for electrophysiology applications. The catheter includes: a tubular member extending along a longitudinal axis from a proximal portion (12A) to a distal portion (12B); an end effector (14) coupled to the distal portion (12B), the end effector (14) including: a plurality of electrodes (37) disposed on the end effector (14); a central loop member (100) extending along the longitudinal axis (L-L) from the distal portion (12B) of the tubular member (12) to a distal portion (100D) of the central loop member (100), the distal portion of the central loop member having a curved portion (100E) connected to two parallel spines (100C, 100F); first and second outer loop members (200, 200′) disposed adjacent the central loop member (100) and on respective sides of the longitudinal axis; each of the first and second outer loop members (200, 200′) extending from the distal portion (12B) of the tubular member (12) to a distal portion (100D) of the central loop member (100), the distal portion of each the first and second outer loop member having a bended portion connected to two parallel spines of each outer loop member, the bended portion including a kidney shaped portion that extends away from the central loop toward the longitudinal axis.

EXAMPLE 2 can be the catheter shown and described with reference to example 1, in which each of the outer loop members (200 or 200′) includes a frame (202 or 202′) comprising first linear segment (202A) connected to a second linear segment (202B) with a first curved segment (C1), the second linear segment (202B) connected to a third linear segment (202C) via a second curved segment (C2), the third linear segment (202C) connected to a distal curved segment (202D) which is connected to a fourth linear segment (202E) which is connected to a fifth linear segment (202F) via a sixth curved segment (C6), the fifth linear segment (202F) connected to a sixth linear segment (202G) via a seventh curved segment (C7).

EXAMPLE 3 can be the catheter shown and described with reference to prior examples, in which the distal curved segment (202D) of each outer loop (202, 202′) comprises a third curved segment (C3) connected to a fourth curved segment (C4) which is connected to a fifth curved segment (C5).

EXAMPLE 4 can be the catheter shown and described with reference to prior examples including example 2, in which the outer loops (200 and 200′) are disposed on each side of the central outer loop (100) so that one outer loop (200) is a mirror image of the other outer loop (200′).

EXAMPLE 5 can be the catheter shown and described with reference to prior examples including example 4, in which a first plane (P1) extends through centers of the outer loops (FIG. 1E) and a second plane (P2) parallel to the first plane (P1) extends through the centers of the central loop.

EXAMPLE 6 can be the catheter shown and described with reference to prior examples including example 5, in which the outer loops and the central loop are contiguous to a third plane (P3) disposed between first and second planes (P1, P2).

EXAMPLE 7 can be the catheter shown and described with reference to prior examples including example 6, in which each of the outer loops (200, 200′) includes a continuous outer frame (202, 202′), each frame comprises the first to sixth linear segments and first to seventh curved segments disposed between the respective first to sixth linear segments.

EXAMPLE 8 can be the catheter shown and described with reference to prior examples including example 6, in which the central loop (100) includes a continuous central frame (102), in which the continuous central frame (102) comprises the first to sixth linear segments with first to seventh curved segments disposed between the respective first to sixth linear segments.

EXAMPLE 9 can be the catheter shown and described with reference to prior examples including example 8, in which a polymer material is disposed over each of the continuous central and outer frames with a plurality of electrodes disposed on an outer surface of the polymer material.

EXAMPLE 10 can be the catheter shown and described with reference to prior examples including example 9, in which the central loop and the outer loops are physically connected at a distal location of the central frame proximate the longitudinal axis (C).

EXAMPLE 11 can be the catheter shown and described with reference to prior examples including example 9, in which the plurality of electrodes is disposed over the outer loops and the central loop so that the electrodes are spaced apart (d) from its adjacent neighbors by a first distance (dy) along the longitudinal axis (dy) and a second distance (dx) along an axis transverse to the longitudinal axis.

EXAMPLE 12: The catheter shown and described with reference to prior examples including example 11, in which the first and second distances (dy, dx) are approximately equal.

EXAMPLE 13 can be the catheter shown and described with reference to example 11, in which the first and second distances (dy, dx) are not equal.

EXAMPLE 14 can be the catheter shown and described with reference to prior examples including example 11, in which each of the outer and central frames has a maximum width of about 0.2 mm and a minimum width of about 0.1 mm with a thickness of about 0.1 mm.

EXAMPLE 15 can be a catheter for electrophysiology applications that includes: (a) a shaft extending along a longitudinal axis to a distal end; and (b) an end effector coupled to the distal end of the shaft, the end effector including: (i) a first loop member disposed on a first side of the longitudinal axis, (ii) a second loop member disposed on a second side of the longitudinal axis, and (iii) a third loop member including: (A) a first spine disposed on the first side of the longitudinal axis, the first spine including a first plurality of electrodes, the first spine is positioned radially outwardly of the first loop member relative to the longitudinal axis, and (B) a second spine disposed on the second side of the longitudinal axis, the second spine including a second plurality of electrodes, the second spine is positioned radially outwardly of the second loop member relative to the longitudinal axis.

EXAMPLE 16 can be the catheter of any of the prior examples in which the third loop member can be symmetrical relative to the longitudinal axis.

EXAMPLE 17 can be the catheter of any of the prior examples in which the first and second loop members each is asymmetrical.

EXAMPLE 18 can be the catheter any of the prior examples in which the first and second loop members is mirror images of each other relative to the longitudinal axis.

EXAMPLE 19 can be the catheter of any of the prior examples in which the third loop member including a distal arch extending between the first and second spines.

EXAMPLE 20 can be the catheter of any of the prior examples in which the third loop member including an elongate structural member at least partially defining each of the first spine, the second spine, and the distal arch.

EXAMPLE 21 can be the catheter of any of the prior examples in which the elongate structural member including nitinol.

EXAMPLE 22 can be the catheter of any of the prior examples in which the elongate structural member including a first linear segment at least partially defining the first spine and a second linear segment at least partially the second spine, the first and second linear segments is integrally formed with each other as a unitary piece.

EXAMPLE 23 can be the catheter of any of the prior examples in which the elongate structural member including first and second linear segments at least partially defining the distal arch, the first and second linear segments is oriented at an obtuse angle relative to each other.

EXAMPLE 24 can be the catheter of any of the prior examples in which the obtuse angle is about 120 degrees.

EXAMPLE 25 can be the catheter of any of the prior examples in which the elongate structural member including a curved segment at least partially defining the distal arch

EXAMPLE 26 can be the catheter of any of the prior examples in which the curved segment having a radius of curvature of about 5.33 mm.

EXAMPLE 27 can be the catheter of any of the prior examples in which the elongate structural member including at least one segment at least partially defining the distal arch, the at least one segment having a varying width.

EXAMPLE 28 can be the catheter of any of the prior examples in which the end effector is configured to transition between an expanded state and a collapsed state.

EXAMPLE 29 can be the catheter of any of the prior examples in which the end effector is resiliently biased to assume the expanded state.

EXAMPLE 30 can be a catheter for electrophysiology applications that include (a) a shaft extending along a longitudinal axis to a distal end; and (b) an end effector coupled to the distal end of the shaft, the end effector including: (i) a first loop member including: (A) a first spine disposed on a first side of the longitudinal axis, the first spine including a first plurality of electrodes, and (B) a second spine disposed on the first side of the longitudinal axis, the second spine including a second plurality of electrodes, (ii) a second loop member including: (A) a third spine disposed on a second side of the longitudinal axis opposite the first side, the third spine including a third plurality of electrodes, and (B) a fourth spine disposed on the second side of the longitudinal axis, the fourth spine including a fourth plurality of electrodes, and (iii) a third loop member including: (A) a fifth spine disposed on the first side of the longitudinal axis, the fifth spine including a fifth plurality of electrodes, the fifth spine is positioned radially outwardly of each of the first and second spines relative to the longitudinal axis, and (B) a sixth spine disposed on the second side of the longitudinal axis, the sixth spine including a sixth plurality of electrodes, the sixth spine is positioned radially outwardly of each of the third and fourth spines relative to the longitudinal axis.

EXAMPLE 31 can be the catheter of any of the prior examples in which the first spine is positioned radially inwardly of the second spine relative to the longitudinal axis, the third spine is positioned radially inwardly of the fourth spine relative to the longitudinal axis.

EXAMPLE 32 can be the catheter of any of the prior examples in which the first and second loop members each is asymmetrical, the third loop member is symmetrical relative to the longitudinal axis.

EXAMPLE 33 can be the catheter of any of the prior examples in which each of the first, second, third, fourth, fifth, and sixth spines is substantially parallel to each other.

EXAMPLE 34 can be a catheter for electrophysiology applications, comprising: (a) a shaft extending along a longitudinal axis to a distal end; and (b) an end effector coupled to the distal end of the shaft, the end effector including: (i) a plurality of loop members, each loop member of the plurality of loop members including a corresponding stress distribution node positioned at a distal portion of the respective loop member, and (ii) a plurality of electrodes affixed to the plurality of loop members.

EXAMPLE 35 can be the catheter of example 34, the plurality of loop members including three loop members.

EXAMPLE 36 can be the catheter of example 35, the plurality of loop members including a pair of outer loop members and an inner loop member positioned radially inwardly of the pair of outer loop members relative to the longitudinal axis.

EXAMPLE 37 can be the catheter of any of examples 34 through 36, each loop member of the plurality of loop members including an elongate structural member.

EXAMPLE 38 can be the catheter of example 37, the elongate structural member of each loop member including the corresponding stress distribution node of the respective loop member.

EXAMPLE 39 can be the catheter of example 37, the stress distribution node of each loop member being affixed to the corresponding elongate structural member of the respective loop member.

EXAMPLE 40 can be the catheter of any of examples 37 through 39, the elongate structural member of each loop member and the corresponding stress distribution node of the respective loop member comprising a same material as each other, and each node is located approximately 3 mm from a transverse axis as referenced to the longitudinal axis.

EXAMPLE 41 can be the catheter of any of examples 37 through 39, the elongate structural member of each loop member and the corresponding stress distribution node of the respective loop member comprising different materials from each other.

EXAMPLE 42 can be the catheter of any of examples 37 through 41, the elongate structural member of each loop member including at least one proximal segment having a first cross dimension, and at least one distal segment positioned distally of the at least one proximal segment and having a second cross dimension greater than the first cross dimension, the at least one distal segment defining the corresponding stress distribution node of the respective loop member. As used herein, the term “cross dimension” includes a sectional measure in one dimension (e.g., width, length, radius) or in two dimensions (sectional area).

EXAMPLE 43 can be the catheter of example 42, the first cross dimension including a first width, the second cross dimension including a second width.

EXAMPLE 44 can be the catheter of example 42, the first cross dimension including a first thickness, the second cross dimension including a second thickness.

EXAMPLE 45 can be the catheter of any of examples 42 through 44, the second cross dimension being about 20% greater than the first cross dimension.

EXAMPLE 46 can be the catheter of any of examples 42 through 45, the elongate structural member of each loop member transitioning from the at least one proximal segment to the at least one distal segment via a stepped configuration.

EXAMPLE 47 can be the catheter of any of examples 42 through 45, the elongate structural member of each loop member transitioning from the at least one proximal segment to the at least one distal segment via a tapered configuration.

EXAMPLE 48 can be the catheter of any of examples 37 through 47, each loop member of the plurality of loop members including a cover disposed about the corresponding elongate structural member.

EXAMPLE 49 can be the catheter of example 48, the cover of each loop member being disposed about the corresponding stress distribution node of the respective loop member.

EXAMPLE 50 can be an end effector of a catheter, the end effector comprising: (a) a plurality of loop members, each loop member of the plurality of loop members including an elongate structural member, each elongate structural member including: (i) at least one proximal segment having a first cross dimension, and (ii) at least one distal segment positioned distally of the at least one proximal segment and having a second cross dimension greater than the first cross dimension; and (b) a plurality of electrodes affixed to the plurality of loop members.

EXAMPLE 51 can be the end effector of example 50, the second cross dimension being about 20% greater than the first cross dimension.

EXAMPLE 52 can be an end effector of a catheter, the end effector comprising: (a) a central loop member extending along a longitudinal axis; (b) a pair of side loop members at least partially disposed on respective sides of the longitudinal axis, each of the side loop members including a corresponding stress distribution node positioned at a distal portion of the respective loop member; and (c) a plurality of electrodes, each of the plurality of electrodes being affixed to a corresponding one of the central or side loop members.

EXAMPLE 53 can be the end effector of example 52, the stress distribution node of each side loop member being defined by an increased width of the respective side loop member

EXAMPLE 54 can be any permutations or combinations of any of the prior Examples.

The embodiments described above are cited by way of example, and the present invention is not limited by what has been particularly shown and described hereinabove. Rather, the scope of the invention includes both combinations and sub combinations of the various features described and illustrated hereinabove, as well as variations and modifications thereof which would occur to persons skilled in the art upon reading the foregoing description and which are not disclosed in the prior art.

IV. Miscellaneous

Any of the instruments described herein may be cleaned and sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, hydrogen peroxide, peracetic acid, and vapor phase sterilization, either with or without a gas plasma, or steam.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A catheter for electrophysiology applications, comprising:
(a) a shaft extending along a longitudinal axis to a distal end; and
(b) an end effector coupled to the distal end of the shaft, the end effector including:
(i) a plurality of loop members, each loop member of the plurality of loop members including a corresponding stress distribution node positioned at a distal portion of the respective loop member, and
(ii) a plurality of electrodes affixed to the plurality of loop members.

2. The catheter of claim 1, the plurality of loop members including three loop members.

3. The catheter of claim 2, the plurality of loop members including a pair of outer loop members and an inner loop member positioned radially inwardly of the pair of outer loop members relative to the longitudinal axis.

4. The catheter of claim 1, each loop member of the plurality of loop members including an elongate structural member.

5. The catheter of claim 4, the elongate structural member of each loop member including the corresponding stress distribution node of the respective loop member.

6. The catheter of claim 5, the stress distribution node of each loop member being affixed to the corresponding elongate structural member of the respective loop member.

7. The catheter of claim 4, the elongate structural member of each loop member and the corresponding stress distribution node of the respective loop member comprising a same material as each other, and each node is located approximately 3 mm from a transverse axis as referenced to the longitudinal axis.

8. The catheter of claim 4, the elongate structural member of each loop member and the corresponding stress distribution node of the respective loop member comprising different materials from each other.

9. The catheter of claim 4, the elongate structural member of each loop member including at least one proximal segment having a first cross dimension, and at least one distal segment positioned distally of the at least one proximal segment and having a second cross dimension greater than the first cross dimension, the at least one distal segment defining the corresponding stress distribution node of the respective loop member.

10. The catheter of claim 9, the first cross dimension including a first width, the second cross dimension including a second width.

11. The catheter of claim 9, the first cross dimension including a first thickness, the second cross dimension including a second thickness.

12. The catheter of claim 9, the second cross dimension being about 20% greater than the first cross dimension.

13. The catheter of claim 9, the elongate structural member of each loop member transitioning from the at least one proximal segment to the at least one distal segment via a stepped configuration.

14. The catheter of claim 9, the elongate structural member of each loop member transitioning from the at least one proximal segment to the at least one distal segment via a tapered configuration.

15. The catheter of claim 4, each loop member of the plurality of loop members including a cover disposed about the corresponding elongate structural member.

16. The catheter of claim 15, the cover of each loop member being disposed about the corresponding stress distribution node of the respective loop member.

17. An end effector of a catheter, the end effector comprising:
(a) a plurality of loop members, each loop member of the plurality of loop members including an elongate structural member, each elongate structural member including:
(i) at least one proximal segment having a first cross dimension, and
(ii) at least one distal segment positioned distally of the at least one proximal segment and having a second cross dimension greater than the first cross dimension; and
(b) a plurality of electrodes affixed to the plurality of loop members.

18. The end effector of claim 17, the second cross dimension being about 20% greater than the first cross dimension.

19. An end effector of a catheter, the end effector comprising:
(a) a central loop member extending along a longitudinal axis;
(b) a pair of side loop members at least partially disposed on respective sides of the longitudinal axis, each of the side loop members including a corresponding stress distribution node positioned at a distal portion of the respective loop member; and
(c) a plurality of electrodes, each of the plurality of electrodes being affixed to a corresponding one of the central or side loop members.

20. The end effector of claim 19, the stress distribution node of each side loop member being defined by an increased width of the respective side loop member.
